(19) United States
(12) Patent Application Publication
(10) Pub. No.: US 2009/0118574 A1
(43) Pub. Date: May 7, 2009

(54) PANTY FOR HEIGHTENING SEXUAL EXPERIENCE DURING COITUS

(76) Inventor: Karen H. Stephenson, Myrtle Beach, SC (US)

Correspondence Address: TILLMAN WRIGHT, PLLC, PO BOX 473909, CHARLOTTE, NC, 28247, US

(21) Appl. No.: 12/266,436

(22) Filed: Nov. 6, 2008

Related U.S. Application Data
(60) Provisional application No. 60/985,956, filed on Nov. 6, 2007.

(51) Int. Cl. A61F 5/00; A41B 9/04; A61H 5/00; A61B 19/00

(52) U.S. Cl. 600/38; 2/406; 128/898; 601/15

(57) ABSTRACT

A panty includes a panty body defining a waist opening of the panty body, the panty body including a crotch portion that extends between and connects a front portion of the panty body with a rear portion of the panty body so as to define two leg openings of the panty body; wherein the crotch portion of the panty body is removably fastened to the front portion of the panty body such that the crotch portion may be detached from the front portion of the panty body.

### CROSS-REFERENCE TO RELATED APPLICATION

The present application is a U.S. nonprovisional patent application of, and claims priority under 35 U.S.C. §119(e) to, U.S. provisional patent application Ser. No. 60/985,956, filed Nov. 6, 2007, which provisional patent application is hereby incorporated herein by reference. Additionally, U.S. patent application publication no. 2008/0108863 is hereby incorporated herein by reference.

### COPYRIGHT STATEMENT

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### BACKGROUND OF THE INVENTION

Eatman U.S. Pat. No. 2,497,443 (“Eatman”) discloses a device for applying heat to the lower abdomen of a woman for alleviating periodic pain without interfering with the normal activities of the woman while the device is worn. The device comprises a V-shaped container made from a rigid material such as aluminum. The device includes a first belt that is secured to the upper ends of the V-shaped container and that is adapted to be secured about the waist of the wearer. The device further includes a second belt that is secured at the apex of the container and is adapted to also surround the body of the wearer below the waist, whereby the two belts together hold the container firmly against the body of the wearer. When worn, the container is oriented with the upper ends of the V-shaped container disposed above the apex of the container. This configuration is generally shown in FIG. 1 of Eatman. In order to heat the lower abdomen, the container is hollow and is adapted to receive and contain hot water. With respect to an alternative embodiment, Eatman discloses that, instead of being water-heated, the device could include an electrical heating element and battery.

Shelton U.S. Pat. No. 6,308,341 (“Shelton”) discloses the combination of an underpant and thermal treatment device for the application of cold or heat therapy and compression to the perineal, genital, and anal areas. More particularly, Shelton discloses an adjustable gel-pack which can apply both compression and thermal treatment to the perineal, genital, and/or anal areas for relief of pain and discomfort. The gel-pack includes connecting straps at each end and an underpant with loops or other attachment means on the front and back external surfaces of the underpants The gel-pack, cooled or heated to a desired temperature, is placed in the genital, perineal, and/or anal areas outside the underpant. One connecting strap is attached to the back external surface of the underpant, and the other connecting strap is pulled forward with as much tension as desired and attached to the front external surface of the underpant, thereby providing temperature and compression treatment to the desired areas for relief of pain and discomfort. The gel-pack is retained in place by the underpant and, specifically, by a waistband that extends about the waist above the hips in an annular fashion that is sufficient to bear the tension produced by the gel-pack and connection straps.

U.S. Pat. No. 5,716,388 (“Petelle”) discloses a flexible fabric pouch for holding a heat transfer pack, the pouch including an adjustable strap for attaching the pouch to a selected part of a person's body for the application of thermal therapy. Petelle discloses that the pouch is preferably made up of a lightweight moisture absorbing fabric such as woven cotton or wool. Petelle further discloses that one side of the pouch may be formed of plural layers of material to provide greater insulation between the thermal pack and the wearer's body, whereby the pouch may be applied to the body in contact therewith on one or the other sides of the pouch depending on the rate of heat transfer desired between the pouch and the wearer's body.

U.S. Pat. No. 6,585,673 (“Bass”) discloses a heat pack positioning device which includes a pocket, a pair of elongated elasticized straps, and a pair of elongated extension straps. The straps are used for securing the heat pack to portion of a person's body as shown in FIGS. 14-17 of Bass.

U.S. Pat. No. 5,948,010 (“Adamec”) discloses a therapeutic heat application device for applying heat to various areas of a person's body. The device includes a pad portion having a wide central portion and a pair of opposed tapered side portions. The pad portion has a shape resembling a pair of lips, and the wide central portion is divided into a plurality of narrow sections by vertically extending seams. A pair of straps extend outwardly from the tapered side portions of the pad portion, and each of the straps have hook and loop fasteners disposed thereon for engaging each other when the straps wrap the pad portion around a body of a wearer. Each of the tapered side portions is divided into two sections by a horizontally extending seam. Adamec further discloses that the sections of the wide central portion and the tapered side portions are filled with cracked corn. Alternatively, Adamec discloses that other natural materials could be used in lieu of the cracked corn, such as dried beans, peas, rice, and even sand.

U.S. Patent Application Publication No. 2002/0193857 (“Lavine”) discloses a therapeutic device comprising an enclosed member and a cover. The cover encompasses the enclosed member and includes a slit for the insertion and removal of the enclosed member. When the enclosed member is removed, the cover can be washed to clean the cover. The enclosed member may comprise a bag and have a plurality of pieces of organic material therein that are capable of being exothermic when heated or endothermic when cooled. Lavine discloses that the bag is preferably filled with feed and/or seed corn, but that the bag alternatively can be filled with rice, cherry pits, or any other seeds, grains and/or fruit pits capable of being exothermic when heated or endothermic when cooled. Lavine broadly discloses that the therapeutic device relieves pain, reduces stress, relaxes muscles, reduces swelling, or provides overall general relaxation and enjoyment.

U.S. Patent Application Publication No. 2006/0137072 (“Visco”) discloses a neck warmer that includes a microwaveable, heat-retaining material, such as buckwheat, rice, or the like.

U.S. Pat. Nos. 5,948,010 and 5,584,086 (“VanWinkel”) discloses a therapeutic pillow used in treating pain. The pillow has a baglike cover filled with natural granular material having a water content in the range of 5% to 25% by weight and a preferred range of water content from 9% to 16%. VanWinkel also discloses that the therapeutic pillow can include different shapes and can be, for example, the shape of a toy for use by children, a pad shape with pockets for inserting hands or feet, or muff-shaped.

Coleman et al. U.S. Patent Application Publication No. 2006/0101558 (hereinafter, “Coleman”) discloses a disposable brief with pockets for thermal inserts. The brief includes exterior front and rear pockets to receive and hold either hot or cold thermal packs at locations such as the lower abdomen, the crotch, the anal area, or the lower back. The brief is adjustable in size such that it can fit men or women. A preferred embodiment is in the form of a diaper having a rear portion that can be slid under a prone patient's back, a crotch portion that can be pulled between the patient's legs, a front portion or panel that can be positioned over the lower abdomen, and securable hip or waist portions such as adjustable straps to secure the brief to the patient and retain the thermal packs in position. The pockets are easily opened and cold or hot packs can be easily inserted or removed from difficult locations, such as behind a patient's back. The disclosed brief can have two or three pockets, which are located at the crotch, at a rear portion, and/or at a front portion.

Cook U.S. Pat. No. 6,308,340 (hereinafter, “Cook”) discloses a pair of women's underpants having an inner panel attached to the inside of a front panel of the underpants. The inner panel is attached such that a pocket is formed inside the underpants. The pocket provides a place for carrying a sanitary item, for example, a sanitary napkin.

Ortmeier U.S. Pat. No. 6,018,823 (hereinafter, “Ortmeier”) discloses a women's undergarment adapted for wearing below a waist of a wearer. The undergarment includes a front portion and a back portion. The back portion includes a pocket formed thereon, i.e. a rear pouch of the undergarment.

Gomez U.S. Pat. No. 6,712,841 (hereinafter, “Gomez”) discloses a panty undergarment with optional pockets for thermal inserts, the purpose of said undergarment being the treatment of hemorrhoids and other similar ailments. The crotch portion of the undergarment may include one or more pocket portions, each capable of holding a swollen tissue treatment device, such as a cold compress or ice pack. The crotch portion may be designed specifically so that the undergarment comfortably fits either a male or female person. The pocket portion may include moisture-absorbent or moisture repellent material. The garment is reusable and washable.

In addition to the foregoing, and while neither disclosed nor suggested by any of the foregoing patent references, it also is known to use warming massage oils and creams in the areas of the genitals of a woman for enhancing a woman's experience during coitus. The use of such oils and creams is believed to increase the chances of a woman experiencing an orgasm. One such cream, which is applied to the clitoris prior to intercourse, is sold under the trademark “FemElle,” further information about which can be found at http://www.femelle.com/sexualenhancement.html.

### SUMMARY OF THE INVENTION

The present invention includes many aspects and features. Moreover, while many aspects and features relate to, and are described in, the context of apparatus and methods for enhancing sexual encounters, the present invention is not limited to use only in apparatus and methods for enhancing sexual encounters, as will become apparent from the following summaries and detailed descriptions of aspects, features, and one or more embodiments of the present invention.

Accordingly, a first aspect of the present invention relates to a panty. The panty includes a panty body defining a waist opening of the panty body, the panty body including a crotch portion that extends between and connects a front portion of the panty body with a rear portion of the panty body so as to define two leg openings of the panty body; wherein the crotch portion of the panty body is removably fastened to the front portion of the panty body such that the crotch portion may be detached from the front portion of the panty body.

In a feature of this aspect of the invention, the crotch portion is permanently secured to the rear portion of the panty body.

In a feature of this aspect of the invention, the crotch portion is integral with and comprises an extension of the rear portion of the panty body.

In a feature of this aspect of the invention, the crotch portion includes a pocket within which a heating pack is removably received and retained.

In a feature of this aspect of the invention, the front portion of the panty body comprises a front panel, wherein the rear portion of the panty body comprises a rear panel, each of the front panel and rear panel being formed from lace that comprises an openwork with figured fabric.

In a feature of this aspect of the invention, the crotch portion comprises a first panel formed from lace that comprises an openwork with figured fabric and a second panel formed from a solid fabric that is attached to the first panel so as to define an interior patch pocket of panty.

In a feature of this aspect of the invention, the panty further comprises a heating pack removably received and retained within the interior patch pocket of the panty.

In a feature of this aspect of the invention, the panty is configured such that, when worn by a woman in the fastened configuration, the crotch portion is located in covering relation to and abutment with the woman's vulva, labia, and clitoris and the heating pack is secured to the woman's vulva, labia, and clitoris for warming thereof.

In a feature of this aspect of the invention, the panty is a boy shorts style of panty.

In a feature of this aspect of the invention, the panty further comprises fringes secured to the panty body around the waste opening and leg openings, each fringe comprising an openwork with figured fabric.

Another aspect of the present invention relates to an apparatus for heightening sexual experience during coitus. The apparatus includes a panty comprising a panty body including, a front portion; a rear portion connected to the front portion so as to define a waste opening of the panty; and a crotch portion extending between and connecting the front portion and the rear portion so as to define two leg openings of the panty body, the crotch portion being removably fastened to the front portion of the panty body on the front of the panty, whereby the crotch portion is detachable from the front portion of the panty body; wherein the crotch portion includes an interior pocket configured to abut and cover a woman's vulva, labia, and clitoris when the panty is worn; and a heating device retained within the crotch pocket whereby, when the panty is worn, the heating device is secured against the woman's vulva, labia, and clitoris.

In a feature of this aspect of the invention, the interior pocket includes a pocket opening located proximate the area of the crotch portion that is removably fastened to the front portion of the panty body.

In a feature of this aspect of the invention, the crotch portion comprises fasteners located proximate an end of the crotch portion, and wherein the crotch portion extends sufficiently from the rear portion such that the end of the crotch portion is located on the front of the panty body when the fasteners secure the end of the crotch portion to the front portion of the panty body, whereby a woman wearing the panty may easily disengage the fasteners without first removing or even repositioning the panty.

In a feature of this aspect of the invention, the crotch portion includes a fastener, wherein the front portion includes a fastener that is configured to removably engage the fastener of the crotch portion, and wherein the fasteners are substantially offset from a middle of the crotch of the panty body when the fasteners are removably engaged with the crotch portion removably fastened to the front portion of the panty body.

Another aspect of the present invention relates to a method. The method includes the steps of providing an apparatus comprising, a panty comprising a panty body including, a front portion; a rear portion connected to the front portion so as to define a waste opening of the panty; and a crotch portion extending between and connecting the front portion and the rear portion so as to define two leg openings of the panty body, the crotch portion being removably fastened to the front portion of the panty body on the front of the panty, whereby the crotch portion is detachable from the front portion of the panty body; wherein the crotch portion includes an interior pocket configured to abut and cover a woman's vulva, labia, and clitoris when the panty is worn; and a heating device retained within the crotch pocket whereby, when the panty is worn, the heating device is secured against the woman's vulva, labia, and clitoris. The method further includes wearing the panty by a woman such that the heating device is retained within the crotch pocket and is secured against the vulva, labia, and clitoris; warming the vulva, labia, and clitoris with the heating device; and unfastening the crotch portion from the front portion of the panty body after the vulva, labia, and clitoris are warmed.

In a feature of this aspect of the invention, the method further includes engaging in sexual intercourse after the crotch portion is unfastened from the front portion of the panty body.

In a feature of this aspect of the invention, the method further includes unfastening the crotch portion from the front portion of the panty body, inserting the heating device within the interior pocket of the crotch portion, and fastening the crotch portion to the front portion.

In a feature of this aspect of the invention, the interior pocket includes a pocket opening located proximate the area of the crotch portion that is removably fastened to the front portion of the panty body.

In a feature of this aspect of the invention, the crotch portion comprises fasteners located proximate an end of the crotch portion, and wherein the crotch portion extends sufficiently from the rear portion such that the end of the crotch portion is located on the front of the panty body when the fasteners secure the end of the crotch portion to the front portion of the panty body, whereby a woman wearing the panty may easily disengage the fasteners without first removing or even repositioning the panty.

In a feature of this aspect of the invention, the crotch portion includes a fastener, wherein the front portion includes a fastener that is configured to removably engage the fastener of the crotch portion, and wherein the fasteners are substantially offset from a middle of the crotch of the panty body when the fasteners are removably engaged with the crotch portion removably fastened to the front portion of the panty body.

Still yet other additional aspects and features of the invention may be found in the incorporated provisional patent application.

In addition to the aforementioned aspects and features of the present invention, it should be noted that the present invention further encompasses the various possible combinations and subcombinations of such aspects and features.

### BRIEF DESCRIPTION OF THE DRAWINGS

One or more preferred embodiments of the present invention now will be described in detail with reference to the accompanying drawings, wherein the same elements are referred to with the same reference numerals, and wherein:

FIG. 1 is a front view of an undergarment, and specifically a panty, in accordance with one or more preferred embodiments of the present invention.

FIG. 1A is digital photograph of a front view of a panty in accordance with one or more preferred embodiments of the present invention, which photograph corresponds to the view illustrated in FIG. 1.

FIG. 2 is a front view of the panty of FIG. 1 in an unfastened configuration.

FIG. 2A is digital photograph of a front view of the panty of FIG. 1A, which photograph corresponds to the view illustrated in FIG. 2A.

FIG. 3 illustrates insertion of a heating pack into an interior patch pocket of the panty of FIG. 1.

FIG. 4 illustrates the panty of FIG. 1 with the heating pack fully inserted into the interior patch pocket.

### DETAILED DESCRIPTION

As a preliminary matter, it will readily be understood by one having ordinary skill in the relevant art (“Ordinary Artisan”) that the present invention has broad utility and application. Furthermore, any embodiment discussed and identified as being “preferred” is considered to be part of a best mode contemplated for carrying out the present invention. Other embodiments also may be discussed for additional illustrative purposes in providing a full and enabling disclosure of the present invention. Moreover, many embodiments, such as adaptations, variations, modifications, and equivalent arrangements, will be implicitly disclosed by the embodiments described herein and fall within the scope of the present invention.

Accordingly, while the present invention is described herein in detail in relation to one or more embodiments, it is to be understood that this disclosure is illustrative and exemplary of the present invention, and is made merely for the purposes of providing a full and enabling disclosure of the present invention. The detailed disclosure herein of one or more embodiments is not intended, nor is to be construed, to limit the scope of patent protection afforded the present invention, which scope is to be defined by the claims and the equivalents thereof. It is not intended that the scope of patent protection afforded the present invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection afforded the present invention is to be defined by the appended claims rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

Furthermore, it is important to note that, as used herein, “a ” and “an” each generally denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at least one of the items,” but does not exclude a plurality of items of the list. Thus, reference to “a picnic basket having cheese or crackers” describes “a picnic basket having cheese without crackers”, “a picnic basket having crackers without cheese”, and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

Referring now to the drawings, one or more preferred embodiments of the present invention are next described. The following description of one or more preferred embodiments is merely exemplary in nature and is in no way intended to limit the invention, its implementations, or uses.

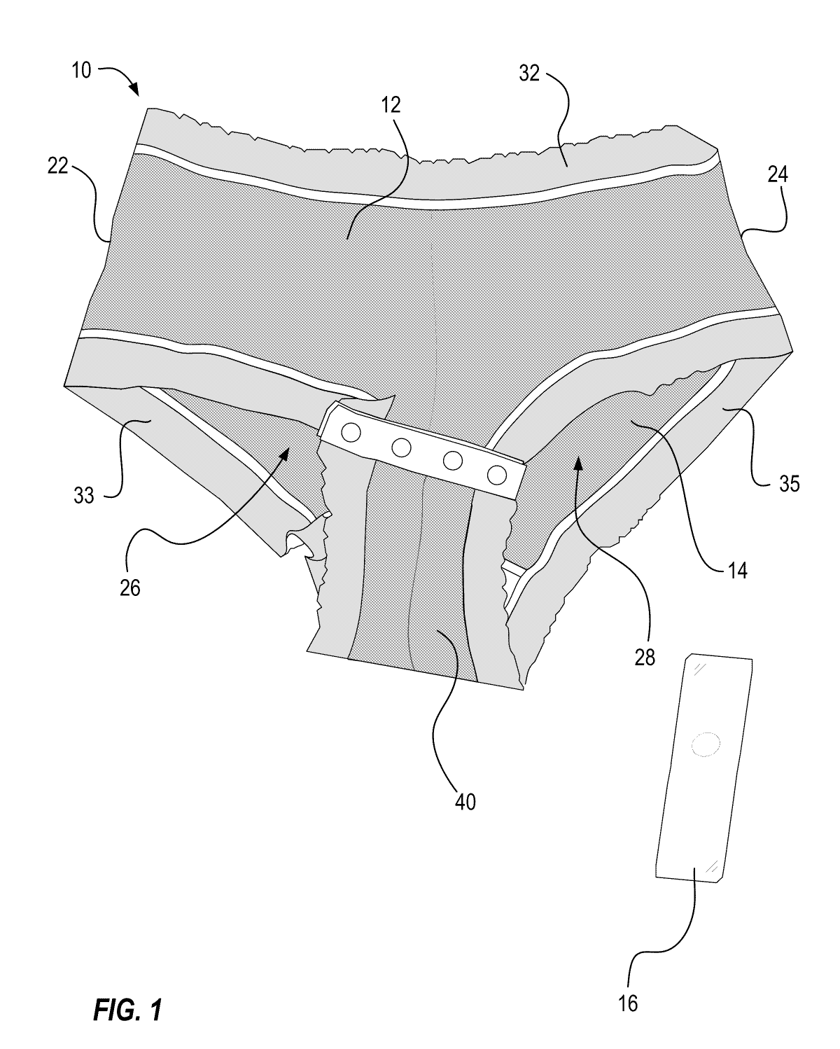

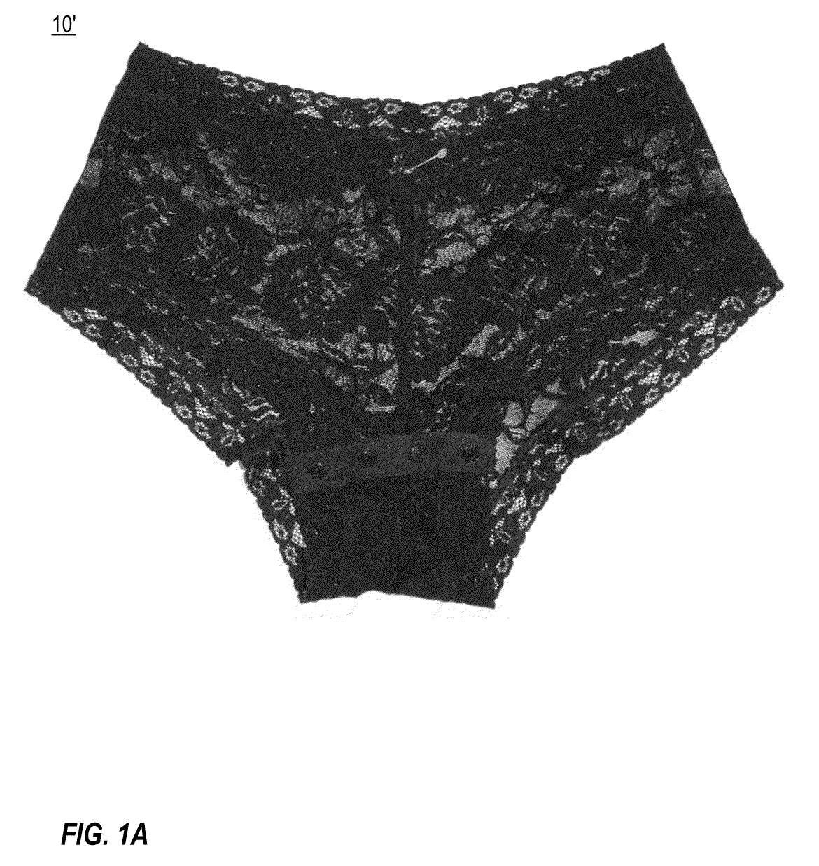

FIG. 1 is a front view of an improved undergarment, and specifically a panty 10, in accordance with a preferred embodiment of the present invention. The panty 10 includes a front panel 12 and a rear panel 14, each of which comprises two smaller panels sewn together respectively along an interior front seam 13 and an interior rear seam 15. The front and rear panels 12,14 are sewn together at a first lateral side 22 and a second lateral side 24 of the panels 12,14, thereby forming an interior seam at each of these sides 22,24.

The panty 10 also includes a crotch panel 40. In the preferred embodiment illustrated, the crotch panel 40 is integrally formed with the rear panel 14 so as to be an extension of the rear panel 14; however, the crotch panel 40 equivalently may be a separate panel that is sewn or otherwise attached to the rear panel 14. In either scenario, the crotch panel 40 is configured and dimensioned to be positioned generally in covering relation to a person's crotch when the panty 10 is worn by a person and is in the fastened configuration, which configuration is illustrated in FIG. 1.

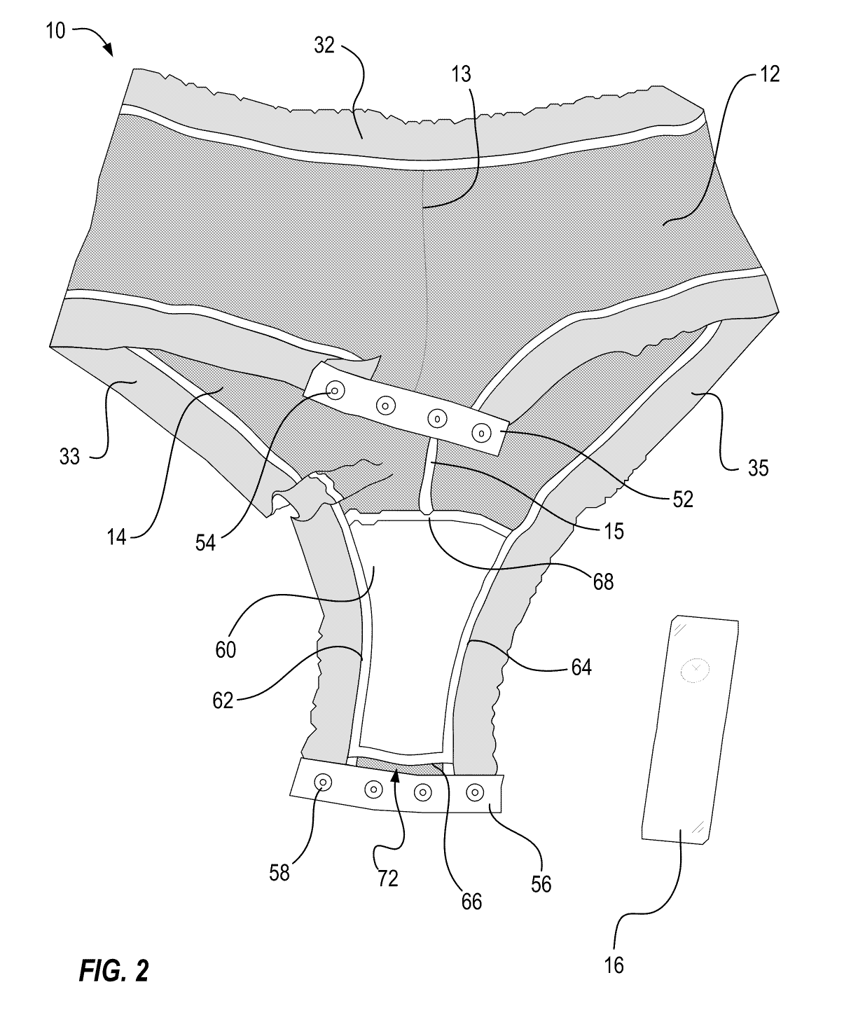

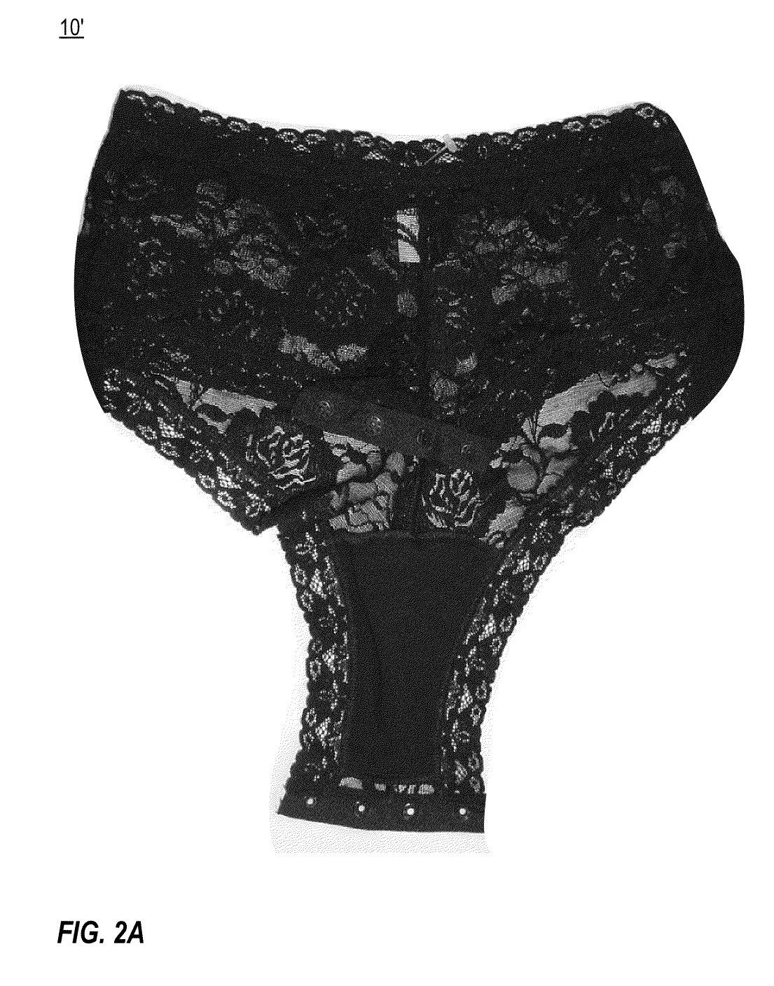

With reference now to FIGS. 2-2A, FIG. 2 is a front view of the panty of FIG. 1 in an unfastened configuration; and FIG. 2A is digital photograph of a front view of the panty of FIG. 1A, which photograph corresponds to the view illustrated in FIG. 2A.

As can be seen in FIG. 2, the front panel 12 includes, attached thereto, a male fastening strip 52 having a plurality of male fasteners 54. Each of these male fasters is configured to mate with a corresponding female fastener 58 of a female fastening strip 56 attached to the crotch panel 40. The panty 10 is thus configured to be transitioned between a fastened configuration, as shown in FIG. 1, and an unfastened configuration, as shown in FIG. 2, by engaging each male fastener 54 with, or disengaging each male fastener 54 from, a respective corresponding female fastener 58. It will thus be understood that the crotch panel 40 can be fastened to and unfastened from the front panel 12.

Additionally, although the illustrated embodiment includes fastening strips 52,56 having a plurality of snap-fasteners 54,58, which snap-fasteners 54,58 are preferably plastic buttons, it will be appreciated that a wide variety of fasteners can be utilized in alternative embodiments. More specifically, metal buttons, hook-and-loop fasteners, or a hook and clasp alternatively may be used in lieu of the plastic snap-fasteners, as could other suitable fasteners known in the art.

It will further be appreciated from the foregoing description that, although the crotch panel 40 is described as part of or attached to the rear panel 14, the crotch panel 40 is releasably fastened to the front panel 12 such that a substantial portion of the crotch panel 40 abuts a front of the person's body when the panty 10 is worn about the person's body. In this respect, the fasteners on the crotch panel 40 are located proximate an end of the crotch panel 40, and the crotch panel extends sufficiently from the rear panel 14 such that the end of the crotch panel 40 is actually located on the front side of the panty 10 when worn, and such that the fasteners are substantially offset from the middle of the crotch so as to be located on the front of the panty 10. Consequently, regardless of the type of fastener or fasteners used, the panty 10 is thereby configured such that a person wearing the panty 10 can easily disengage the fastener or fasteners without first removing the panty 10, and in fact, without even repositioning the panty 10.

With further reference to FIG. 1, the panty 10 also includes a fringe 32 that extends along and is sewn to a top portion of the front and rear panels 12,14 so as to form a waistband of the panty 10. A seam is illustrated in the drawings where the fringe 32 is sewn to the panels 12,14. Also as shown in FIG. 1, where the panty 10 is illustrated in a fastened configuration (which configuration is further described hereinbelow), a similar fringe 33 extends along and is sewn to a bottom portion of the front and rear panels 12,14 so as to form a first leg band having a first leg opening 26 of the panty 10, and a similar fringe 35 extends along and is sewn to a bottom portion of the front and rear panels 12,14 so as to form a second leg band having a second leg opening 28 of the panty 10.

The panty 10 is configured and dimensioned to be worn about a person's lower trunk such that the waistband of the panty 10 is generally located at or below the person's waist, and further, when in the fastened configuration, such that the person's legs pass through the leg openings 26,28.

Returning to FIG. 2, the crotch panel 40 further includes, attached thereto, a second panel 60 that is sewn onto it such that an interior patch pocket is defined by the crotch panel 40 and the second panel 60. As can be seen in FIG. 2, the second panel 60 is sewn onto the crotch panel 40 at a left edge 62 and a right edge 64 of the second panel 60 generally along seams joining the fringe 32 to the crotch panel 40. Unlike the left and right edges 62,64, a bottom edge 66 of the second panel 60 is not sewn onto the crotch panel 40, and thus a pocket opening 72, which provides access to the interior patch pocket, is defined by the bottom edge 66 of the second panel 60 and the crotch panel 60. A top edge 68 of the second panel 60 is sewn onto the crotch panel 40, however, unlike the left and right edges 62,64, preferably only an approximate midpoint of the top edge 68 is sewn into the crotch panel 40 at seam 15. It will be appreciated that this midpoint attachment of the second panel 60 serves to prevent objects of a sufficient size inserted through the pocket opening 72 into the interior patch pocket from passing through the opposite side of the interior patch pocket corresponding to the top edge 68 of the second panel 60. The panty 10 is preferably configured such that when the panty 10 is worn by a woman in the fastened configuration, the second panel 60 is located in covering relation to and abutment with the woman's genitals, and specifically, in covering relation to and abutment with the woman's vulva, labia, and clitoris. As such, the second panel 60 preferably comprises a soft cotton fabric and is solid, i.e., is not an openwork.

The interior patch pocket is configured to receive therein a heating pack 16. Moreover, the heating pack 16 preferably is sized and dimensioned to correspond to the size of the interior patch pocket. The heating pack 16 is configured to provide heat and/or a warmth. Preferably, the heating pack 16 is a chemical heat pad, such as, for example, a sodium acetate heat pad. It is well known that a sodium acetate heat pad can be activated by bending or flexing a flat disc of metal suspended therein and that, after use, such a pad can be re-activated by placement in boiling water for ten to fifteen minutes.

In alternative embodiments, a heat transfer pack is utilized instead. A heat transfer pack is a container filled with a material having a high specific heat capacity. For example, a container is first filled with rice, buckwheat, flax seed, or another naturally granular material. The container preferably includes a plurality of ribbed portions, each ribbed portion being filled with the naturally granular material. Such a heat transfer pack is then heated in a microwave for one to two minutes, after which time a moist heat emanates from the pack.

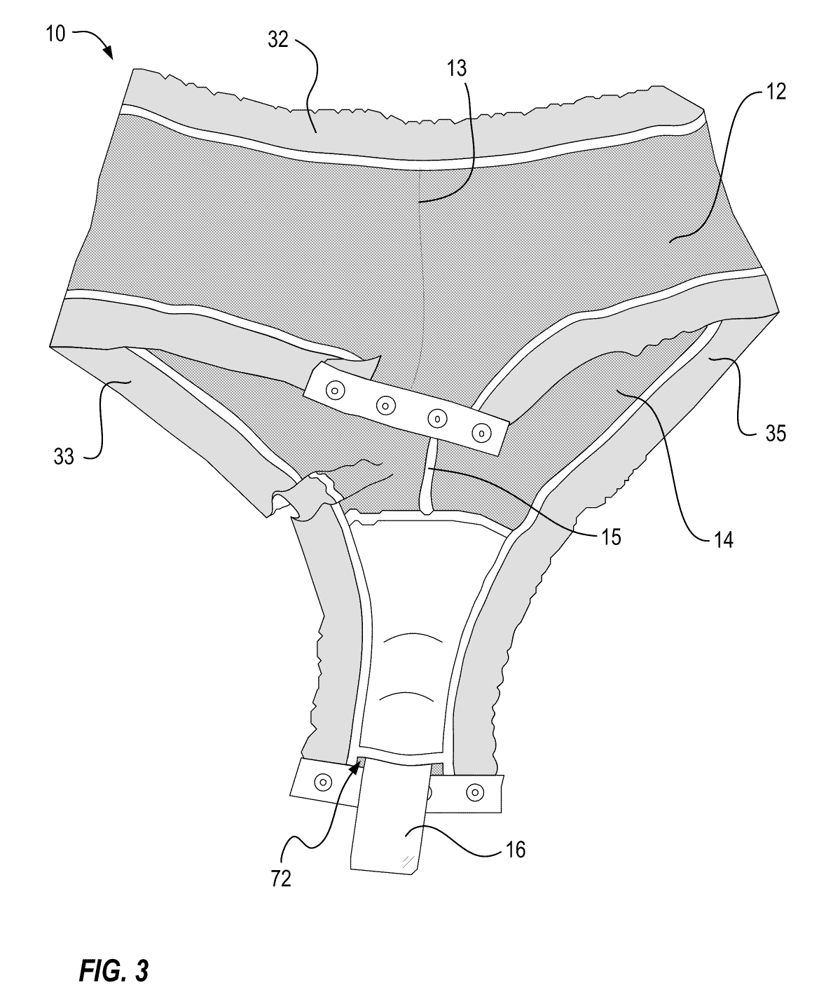

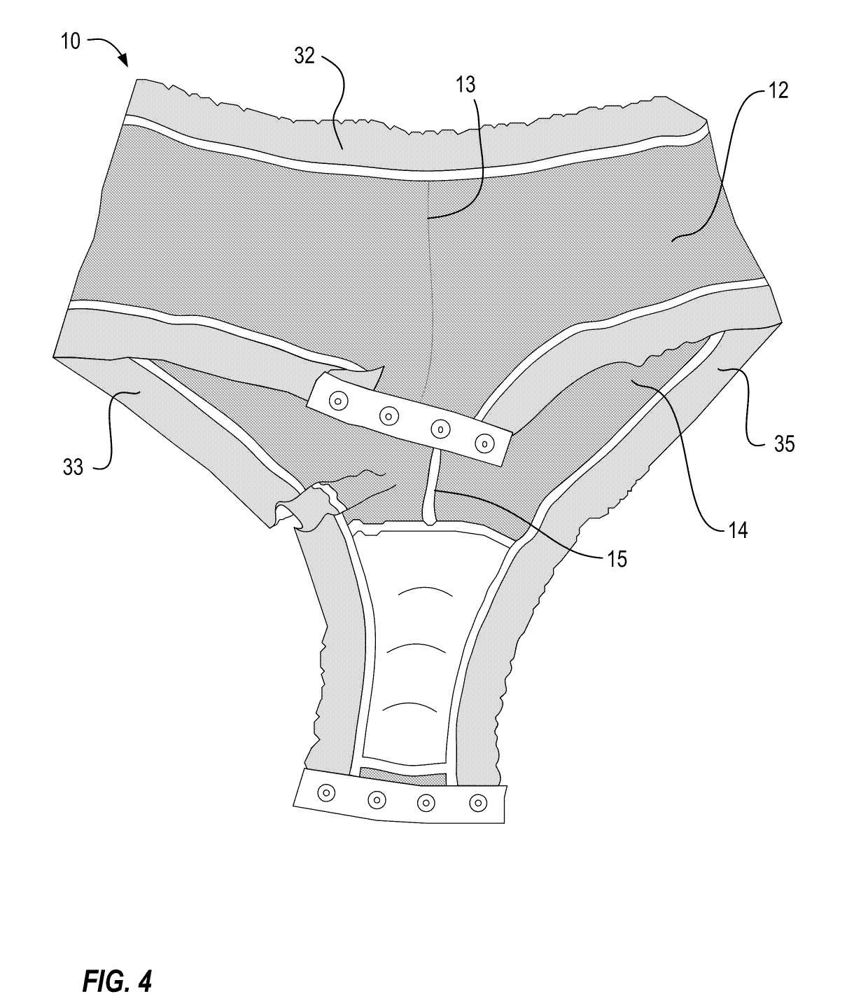

Regardless of the type of heating pack utilized, the heating pack is dimensioned and configured to be received within the interior patch pocket such that, when the panty 10 is worn by a woman in the fastened configuration, the heating pack is secured against the woman's genitals, and specifically, secured against the woman's vulva, labia, and clitoris whereby the vulva, labia, and clitoris are warmed by the heating pack. FIGS. 3-4 illustrate the heating pack 16 being inserted into the interior patch pocket of the panty 10. After insertion of the heating pack 16, the panty 10 may be transitioned back into the fastened configuration, all while being worn by a woman.

The front and rear panels 12,14 can be made from any of a wide variety of fabrics or materials suitable for undergarment construction, such as, for example, cotton, spandex, polyester, lace, or a blend thereof. In a preferred embodiment, the front and rear panels 12,14 are made from a blend of 65% cotton, 20% spandex, and 15% polyester. The fabric or material can additionally include a decorative design, such as, for example a pattern of roses or other flowers. The fabric or material preferably is soft, pliable, and elastically stretchable.

Preferably, the front and rear panels 12,14 are formed from lace that comprises an openwork with figured fabric. In this respect, FIG. 1A is a digital photograph of a front view of a panty 10′ in accordance with one or more preferred embodiments of the present invention, which photograph corresponds to the view illustrated in FIG. 1. The panty 10′ of FIG. 1A includes lace that comprises an openwork with figured fabric depicting decorative roses.

Like the front and rear panels 12,14, each fringe 32,33,35 can be made from any of a wide variety of fabrics or materials suitable for undergarment construction, such as, for example, cotton, spandex, polyester, lace, or a blend thereof, and can be made from the same fabric or material as the front and rear panels 12,14 or from a different fabric or material. In a preferred embodiment, each fringe 32,33,35 is made from a blend of 65% cotton, 20% spandex, and 15% polyester. The fabric or material can additionally include a decorative design, such as, for example a pattern of roses or other flowers. The fabric or material preferably is soft, pliable, and elastically stretchable.

Preferably, each fringe 32,33,35 is formed from lace that comprises an openwork with figured fabric. In this respect, in FIG. 1A the panty 10′ includes a waistband and two leg bands each comprising an openwork with figured fabric depicting decorative flowers.

The description above and FIGS. 1-4 describe and illustrate a panty 10 of the “boy shorts” variety. Although described and illustrated in such a style, it will be appreciated that alternative styles of panties could be utilized in accordance with the invention, including, for example, a bikini style, a brief style, or a thong style of panty.

A method of use in accordance with one or more preferred embodiments of the present invention now is described.

First, a woman puts on the panty 10. The heating pack 16 then is activated such that heat emanates from the heating pack 16. If the panty 10 is being worn in its fastened configuration, then the undergarment is next transitioned to the unfastened configuration by unfastening the crotch panel 40 from the front panel 12. Once in the unfastened configuration, the heating pack 16 is inserted into the interior patch pocket defined between the crotch panel 40 and the second panel 60 via the pocket opening 72. The panty 10 is then transitioned back into the fastened configuration. It will be appreciated that at this stage the woman will feel heat and/or warmth radiating from the heating pack 16 into her pelvic region and, specifically, will experience warming of the vulva, labia, and clitoris.

The undergarment preferably is worn in the fastened configuration for ten to twenty minutes, during which time the heat will increase blood flow to the woman's genitals. Specifically, the heat from the heat pack will cause the vulva to warm and, in particular, the labia and clitoris to warm. It is believed that the increased blood flow will heighten sensitivity and enhance any subsequent or concurrent sexual encounter. Warm moist heat also is believed to have a relaxing and comforting effect that can be enjoyed during this time. Moreover, this period of time provides time for foreplay, if desired.

Following heating of the pelvic region, the panty 10 preferably is transitioned to the unfastened configuration so that the panty 10 will not inhibit or otherwise interfere with coitus.

The panty 10 is specifically designed to be worn by a woman for the purposes of sexual enhancement. However, it also is believed that, during coitus, a male partner also will experience a warming, pleasurable sensation with his penis upon penetration as a result of use of the panty 10 of the present invention.

As will be appreciated, after use the panty 10 can be cleaned, if necessary, by removal of the heating pack 16 and washing. The heat transfer pack similarly can be reused, either by boiling in water to reset a chemical heating pack or by simply reheating a heat transfer pack in a microwave oven.

As further will be appreciated, use of the panty 10 in accordance with an aspect of the present invention does not result in any oil stains or other mess requiring cleaning. Unlike oils and creams, the panty 10 itself does not necessitate cleaning.

Based on the foregoing description, it will be readily understood by those persons skilled in the art that the present invention is susceptible of broad utility and application. Many embodiments and adaptations of the present invention other than those specifically described herein, as well as many variations, modifications, and equivalent arrangements, will be apparent from or reasonably suggested by the present invention and the foregoing descriptions thereof, without departing from the substance or scope of the present invention.

Accordingly, while the present invention has been described herein in detail in relation to one or more preferred embodiments, it is to be understood that this disclosure is only illustrative and exemplary of the present invention and is made merely for the purpose of providing a full and enabling disclosure of the invention. The foregoing disclosure is not intended to be construed to limit the present invention or otherwise exclude any such other embodiments, adaptations, variations, modifications or equivalent arrangements, the present invention being limited only by the claims appended hereto and the equivalents thereof.

What is claimed is:

1. A panty, comprising:
(a) a panty body defining a waist opening of the panty body, the panty body including a crotch portion that extends between and connects a front portion of the panty body with a rear portion of the panty body so as to define two leg openings of the panty body;
(b) wherein the crotch portion of the panty body is removably fastened to the front portion of the panty body such that the crotch portion may be detached from the front portion of the panty body.

2. The panty of claim 1, wherein the crotch portion is permanently secured to the rear portion of the panty body.

3. The panty of claim 1, wherein the crotch portion is integral with and comprises an extension of the rear portion of the panty body.

4. The panty of claim 1, wherein the crotch portion includes a pocket within which a heating pack is removably received and retained.

5. The panty of claim 1, wherein the front portion of the panty body comprises a front panel, wherein the rear portion of the panty body comprises a rear panel, each of the front panel and rear panel being formed from lace that comprises an openwork with figured fabric.

6. The panty of claim 5, wherein the crotch portion comprises a first panel formed from lace that comprises an openwork with figured fabric and a second panel formed from a solid fabric that is attached to the first panel so as to define an interior patch pocket of panty.

7. The panty of claim 6, further comprising a heating pack removably received and retained within the interior patch pocket of the panty.

8. The panty of claim 7, wherein the panty is configured such that, when worn by a woman in the fastened configuration, the crotch portion is located in covering relation to and abutment with the woman's vulva, labia, and clitoris and the heating pack is secured to the woman's vulva, labia, and clitoris for warming thereof.

9. The panty of claim 1, wherein the panty is a boy shorts style of panty.

10. The panty of claim 1, further comprising fringes secured to the panty body around the waste opening and leg openings, each fringe comprising an openwork with figured fabric.

11. An apparatus for heightening sexual experience during coitus, comprising:
(a) a panty comprising a panty body including,
(i) a front portion;
(ii) a rear portion connected to the front portion so as to define a waste opening of the panty; and
(iii) a crotch portion extending between and connecting the front portion and the rear portion so as to define two leg openings of the panty body, the crotch portion being removably fastened to the front portion of the panty body on the front of the panty, whereby the crotch portion is detachable from the front portion of the panty body;
(iv) wherein the crotch portion includes an interior pocket configured to abut and cover a woman's vulva, labia, and clitoris when the panty is worn; and
(b) a heating device retained within the crotch pocket whereby, when the panty is worn, the heating device is secured against the woman's vulva, labia, and clitoris.

12. The apparatus of claim 11, wherein the interior pocket includes a pocket opening located proximate the area of the crotch portion that is removably fastened to the front portion of the panty body.

13. The apparatus of claim 11, wherein the crotch portion comprises fasteners located proximate an end of the crotch portion, and wherein the crotch portion extends sufficiently from the rear portion such that the end of the crotch portion is located on the front of the panty body when the fasteners secure the end of the crotch portion to the front portion of the panty body, whereby a woman wearing the panty may easily disengage the fasteners without first removing or even repositioning the panty.

14. The apparatus of claim 11, wherein the crotch portion includes a fastener, wherein the front portion includes a fastener that is configured to removably engage the fastener of the crotch portion, and wherein the fasteners are substantially offset from a middle of the crotch of the panty body when the fasteners are removably engaged with the crotch portion removably fastened to the front portion of the panty body.

15. A method, comprising the steps of:
(a) providing an apparatus comprising,
(i) a panty comprising a panty body including,
(A) a front portion;
(B) a rear portion connected to the front portion so as to define a waste opening of the panty; and
(C) a crotch portion extending between and connecting the front portion and the rear portion so as to define two leg openings of the panty body, the crotch portion being removably fastened to the front portion of the panty body on the front of the panty, whereby the crotch portion is detachable from the front portion of the panty body;
(D) wherein the crotch portion includes an interior pocket configured to abut and cover a woman's vulva, labia, and clitoris when the panty is worn; and
(ii) a heating device retained within the crotch pocket whereby, when the panty is worn, the heating device is secured against the woman's vulva, labia, and clitoris;
(b) wearing the panty by a woman such that the heating device is retained within the crotch pocket and is secured against the vulva, labia, and clitoris;
(c) warming the vulva, labia, and clitoris with the heating device; and
(d) unfastening the crotch portion from the front portion of the panty body after the vulva, labia, and clitoris are warmed.

16. The method of claim 15, further comprising engaging in sexual intercourse after the crotch portion is unfastened from the front portion of the panty body.

17. The method of claim 15, further comprising unfastening the crotch portion from the front portion of the panty body, inserting the heating device within the interior pocket of the crotch portion, and fastening the crotch portion to the front portion.

18. The method of claim 15, wherein the interior pocket includes a pocket opening located proximate the area of the crotch portion that is removably fastened to the front portion of the panty body.

19. The method of claim 15, wherein the crotch portion comprises fasteners located proximate an end of the crotch portion, and wherein the crotch portion extends sufficiently from the rear portion such that the end of the crotch portion is located on the front of the panty body when the fasteners secure the end of the crotch portion to the front portion of the panty body, whereby a woman wearing the panty may easily disengage the fasteners without first removing or even repositioning the panty.

20. The method of claim 15, wherein the crotch portion includes a fastener, wherein the front portion includes a fastener that is configured to removably engage the fastener of the crotch portion, and wherein the fasteners are substantially offset from a middle of the crotch of the panty body when the fasteners are removably engaged with the crotch portion removably fastened to the front portion of the panty body.
